(19) United States
(12) Patent Application Publication
(10) Pub. No.: US 2006/0083702 A1
(43) Pub. Date: Apr. 20, 2006

(54) Hair fiber treating method

(76) Inventors: Thomas Fondin, Taverny (FR); Anne Sabbagh, Rueil Malmaison (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/097,120

(22) Filed: Apr. 4, 2005

Related U.S. Application Data
(60) Provisional application No. 60/571,919, filed on May 18, 2004.

(30) Foreign Application Priority Data
Apr. 2, 2004 (FR) 04 50668

(51) Int. Cl. A61K 8/46

(52) U.S. Cl. 424/70.2

(57) ABSTRACT

The present disclosure relates to a hair fiber treatment method without a hair fixing step. This method includes applying a reducing composition containing no ceramide onto hair fibers, and raising the temperature of the hair fibers with a heating iron at a temperature of at least 60° C. The reducing composition includes at least one reducing agent and at least one non polymeric cosmetic active agent. The at least one reducing agent is selected from thiol group containing compounds.

This application claims benefit of U.S. Provisional Application No. 60/571,919, filed May 18, 2004, and French Patent Application No. 04 50668, filed Apr. 2, 2004, all of which are hereby incorporated by reference.

The present disclosure relates to a method for treating hair fiber.

Several methods for obtaining the permanent reshaping of hair are known. A typical method for hair reshaping includes a series of steps, including: a first step, wherein keratin disulfide bonds (cystine) are opened with a composition containing a reducing agent; an optional rinsing step; and a third step, wherein the disulfide bonds are reformed by applying an oxidizing composition (fixing solution) to the hair after the hair has been straightened or placed under tension by a suitable means, e.g. curlers or the like, so as to give the hair a desired form. This method allows the hair to attain a waved, uncurled, backcombed, or straightened configuration.

Generally, reducing agent compositions that can be used to carry out the first step of these known methods include thiol group containing compounds, such as thioglycolic acid, cysteine, cysteamine, thiolactic acid and glycerol monothioglycolate.

In addition to the above described method, hair reshaping methods utilizing heat treatment of the hair fibers are known. For example, Patent application JP 2000 256 146 describes a method for permanently reshaping hair, including the application of a cosmetic composition containing from 2 to 11% reducing agents and from 0.2 to 4% diammonium dithiodiglycolate. After applying the reducing composition, a the temperature of the hair is increased with a heating iron at a temperature from 60-220° C.

The above described methods are not completely satisfactory however. While these methods allow the hair to be reshaped very efficiently, they inflict substantial damage on the hair fibers. Methods requiring the use of a heating iron also require a post-iron fixing step as well, which increases the treatment time. Further, the shape of the hair that results from the use of the above methods is irreversible. As a result, the contrast between the treated portion of the hair and the roots of the hair is highly visible as the hair regrows. Finally, if the treatment is conducted on colored hair, it often causes the hair color to fade as a result of the treatment.

Thus, one embodiment of the present disclosure provides a hair fiber treating method that can compensate for the drawbacks of the prior art.

More specifically, one embodiment of the present disclosure is a hair fiber treating method that enables changes to be made to the hair fiber behavior, while limiting the damage caused to the hair. The method according to this embodiment can also control the volume of hair, and can enhance the cosmetic benefits provided to the hair by the process. These benefits include but are not limited to softness, shine, and ease of combing. In addition, this method can improve the preservation of colored hair shades, and can preserve the natural hair aspect (the contrast between the treated portions of the hair and the roots of the hair).

According to another embodiment of the disclosure is a hair fiber treatment method that has one or more of reduced treatment time and long-lasting results.

The inventors have discovered a hair treatment method that overcomes the drawbacks of the prior art and allows the above aspects to be obtained. According to one embodiment, the hair treatment method does not include a hair fixing step, and comprises applying at least one reducing composition that contains no ceramide to the hair. The at least one reducing composition includes at least one reducing agent and at least one cosmetic active agent. The at least one reducing agent is selected from thiol group containing compounds, and the at least one cosmetic active agent is selected from non-polymeric active agents. The method also includes a hair fiber temperature rising step, wherein the hair is heated with a heating iron at a temperature of at least 60° C. This hair fiber temperature raising step may be performed before or after an optional hair fiber rinsing step.

Accordingly, one embodiment of the present disclosure is a hair fiber treating method without hair fixing, comprising:
applying at least one reducing composition containing no ceramide onto hair fibers, the reducing composition comprising at least one reducing agent selected from thiol group containing compounds and at least one cosmetic active agent selected from non-polymeric active agents;
raising the temperature of the hair fibers with a heating iron at a temperature of at least 60° C., either before or after an optional hair fiber rinsing step.

As used herein, ‘without hair fixing’ means without any additional application of a composition comprising an oxidizing agent, for example, hydrogen peroxide or a bromate.

In a non-limiting embodiment of the disclosure, the at least one reducing composition does not comprise dithiodiglycolic acid or any salt thereof.

The at least one cosmetic non-polymeric active agent is chosen from waxes, anionic, cationic, non-ionic, amphoteric or zwitterionic surfactants, active agents combating hair loss, anti-dandruff agents, sequestering agents, opacifying agents, dyes, sunscreen agents, vitamins, provitamins, fatty acids, fatty alcohols, esters, pearlescent pigments, mineral, vegetable or synthetic oils, fragrances and preserving agents, and combinations thereof.

Vitamins or provitamins that may be used as cosmetic active agents in the reducing composition include vitamins A, B3 (niacinamide), B5, C, E, F and provitamin B5.

Sunscreen agents that may be used as suitable cosmetic active agents include organic and mineral sunscreen.

Suitable organic sunscreens include those selected from cinnamic derivatives, dibenzoylmethane derivatives, salicylic derivatives, camphor derivatives, triazine derivatives (such as those described in patent applications U.S. Pat. No. 4,367,390, EP 0 863 145, EP 0 517 104, EP 0 570 838, EP 0 796 851, EP 0 775 698, EP 0 878 469, EP 0 933 376 and EP 0 893 119), benzophenone derivatives, β,β′-diphenylacrylate derivatives, benzimidazole derivatives, bis-benzoazolyle derivatives (such as those described in patents EP 0 669 323 and U.S. Pat. No. 2,463,264), bis-(hydroxyphenylbenzotriazole) methylene derivatives (such as those described in patent applications U.S. Pat. No. 5,237,071, U.S. Pat. No. 5,166,355, GB-2303549, DE-19726184 and EP 0 893 119), p-aminobenzoic acid derivatives, dimers derived from α-alkyl styrene (such as those described in patent application DE-1 9855649).

Suitable mineral sunscreens include those selected from pigments or nanopigments (the average particle diameter of which typically ranges from 5 nm to 100 nm, preferably from 10 nm to 50 nm) of coated or uncoated metallic oxides, such as titanium oxide nanopigments (amorphous or crystallized as rutile and/or anatase), oxides of iron, oxides of zinc, oxides of zirconium, or oxides of cerium, all of which are well known ultraviolet photoprotective agents. Suitable coating agents for the coated metallic oxides include alumine and/or aluminum stearate. Such metallic oxide nanopigments, whether coated or uncoated, are described in particular, in patent applications EP 0 518 772 and EP 0 518 773.

Fatty acids that are suitable for use as non-polymeric active agents are chosen from one or more of, for example, saturated or unsaturated, linear or branched C₈-C₃₀ carboxylic acids, such as palmitic acid, oleic acid, linoleic acid, myristic acid, stearic acid, and lauric acid.

Fatty alcohols that are suitable include one or more of, for example, saturated or unsaturated, linear or branched C₈-C₃₀ alcohols, such aspalmityl, oleyl, linoleyl, myristyl, stearyl and lauryl alcohols.

Anionic surfactants that are suitable include one or more of the salts of following compounds (in particular alkali salts, especially sodium, ammonium, amine salts, amino alcohols salts or magnesium salts): alkylsulfates, alkyl ethersulfates, alkylamidoethersulfates, alkylarylpolyether sulfates, monoglyceride sulfates, alkylsulfonates, alkylphosphates, alkylamidesulfonates, alkylarylsulfonates, α-olefin-sulfonates, paraffin-sulfonates; alkyl sulfosuccinates, alkyl ether sulfosuccinates, alkyl amide sulfosuccinates; alkylsulfosuccinamates; alkylsulfoacetates; alkyletherphosphates; acylsarcosinates; acylisethionates; N-acyltaurates; alkyl or acyl moieties of the aforementioned compounds comprising, for example, from 12 to 20 carbon atoms; and aryl moieties (preferably a phenyl or benzyl moiety) of the aforementioned compounds. Other suitable anionic surfactants include fatty acid salts (such as oleic, ricinoleic, palmitic, stearic acid salts); coconut oil or hydrogenated coconut oil acids; and acyl-lactylates the acyl moiety of which comprises from 8 to 20 carbon atoms. Weakly anionic surfactants may also be used. These weakly anionic surfactants include: alkyl D galactoside uronic acids and the salts thereof; polyoxyalkylene carboxylic ether acids and salts thereof, in particular those comprising from 2 to 50 ethylene oxide moieties; and mixtures thereof. Anionic surfactants of the acid or polyoxyalkylene carboxylic ether type are, for example, those having the following formula (1):
R₁—(OC₂H₄)ₙ—OCH₂COOA

wherein:

R₁ is chosen from alkyl, alkylamido and alkaryl moieties; n is an integer or a decimal (mean value) ranging from 2 to 24, such as from 3 to 10, the alkyl moiety having, for example, 6 to 20 carbon atoms, and aryl corresponding, for example, to a phenyl group; and A is H, ammonium, Na, K, Li, Mg or a monoethanolamine or triethanolamine rest. Mixtures of more than one compound of formula (1) may also be used, such as mixtures in which R₁ moieties are different.

Compounds of formula (1) are commercially marketed, for example by CHEM Y, under trade names AKYPOS (NP40, NP70, OP40, OP80, RLM25, RLM38, RLMQ 38 NV, RLM 45, RLM 45 NV, RLM 100, RLM 100 NV, RO 20, RO 90, RCS 60, RS 60, RS 100, RO 50) or by SANDOZ under trade names SANDOPAN (DTC Acid, DTC).

Non-ionic surfactants that are suitable in the present disclosure may be chosen from at least one of polyethoxylated, polypropoxylated or polyglycerolated fatty alcohols, polyethoxylated, polypropoxylated or polyglycerolated fatty alpha-diols, polyethoxylated, polypropoxylated or polyglycerolated fatty alkylphenols and polyethoxylated, polypropoxylated or polyglycerolated fatty acids, all having a fatty chain comprising from 8 to 18 carbon atoms, where the number of ethylene oxide or propylene oxide moieties may be up to 2 to 50, and the number of glycerol moieties may be up to 2 to 30. Other suitable non-ionic surfactants include ethylene oxide and propylene oxide copolymers; ethylene oxide and propylene oxide condensation products onto fatty alcohols; polyethoxy fatty amides, including, for example, those with 2 to 30 moles of ethylene oxide; polyglycerol fatty amides having, for example, an average of 1 to 5 glycerol moieties, such as from 1.5 to 4 glycerol moieties; fatty acid esters of oxyethylene sorbitan, including, for example, those with 2 to 30 moles ethylene oxide; saccharose fatty acid esters; polyethyleneglycol fatty acid esters; alkyl polyglycosides; N-alkyl glucamine derivatives; amine oxides, including (C₁₀-C₁₄) alkyl amine oxides; and N-acylaminopropyl morpholine oxides.

Amphoteric or zwitterionic surfactants that are suitable include one or more of aliphatic secondary or tertiary amine derivatives, where the aliphatic moiety is a linear or a branched chain with from 8 to 18 carbon atoms and has at least one anionic water solubilizing group (for example carboxylate, sulfonate, sulfate, phosphate or phosphonate). One or more of alkyl (C₈-C₂₀) betaines, sulfo betaines, alkyl (C₈-C₂₀) amidoalkyl (C₁-C₆) betaines or alkyl (C₈-C₂₀) amidoalkyl (C₁-C₆) sulfobetaines may also be used.

Suitable amine derivatives include one or more of the products commercially marketed under the trade name MIRANOL, such as those described in U.S. Pat. Nos. 2,528,378 and 2,781,354 and registered in the CTFA dictionary, 3d edition, 1982, under the denominations Amphocarboxyglycinates and Amphocarboxypropionates having the following structures respectively:
R₂—CONHCH₂CH₂—N⁺(R₃)(R₄)(CH₂COO⁻) (2)

wherein: R₂ is chosen from alkyl moieties of an R₂—COOH acid that are present in hydrolyzed coconut oil, heptyl, nonyl and undecyl moieties, R₃ is a beta-hydroxyethyl moiety and R₄ a carboxymethyl moiety;

and
R₂′—CONHCH₂CH₂—N(B)(Z′) (3)

wherein:

B represents —CH₂CH₂OX′, Z′ represents —(CH₂)_{z}—Y′, where z=1 or 2,

X′ is a —CH₂CH₂—COOH moiety or a hydrogen atom;

Y′ is —COOH or —CH₂—CHOH—SO₃H moiety;

R₂′ is chosen from alkyl moieties of an R₉—COOH acid that is present in the coconut oil or in hydrolysed linseed oil, alkyl moieties, including moieties containing a C₇, C₉, C₁₁ or C₁₃, a C₁₇ and their iso forms, or an unsaturated C₁₇.

A non-limiting example of a suitable amine derivative is Cocoamphocarboxyglycinate, which is commercially marketed under the trade name MIRANOL C2M concentrate by MIRANOL.

Cationic surfactants that are suitable include: primary, secondary or tertiary fatty amine salts; polyoxyalkylene amines; quaternary ammonium salts such as tetraalkylammonium, alkylamidoalkyltrialkylammonium, trialkylbenzylammonium, trialkylhydroxyalkyl-ammonium or alkylpyridinium chlorides or bromides; imidazoline derivatives; and cationic amine oxides.

Non-limiting examples of vegetable oils that are suitable include: sweet almond oil, avocado oil, castor oil, olive oil, jojoba oil, sunflower oil, wheat germ oil, sesame oil, groundnut oil, grape seed oil, soybean oil, rapeseed oil, safflower oil, coconut oil, corn oil, hazelnut oil, shea butter, palm oil, apricot kernel oil, calophyllum oil, and mixtures thereof.

Non-limiting examples of suitable synthetic oils include polyolefins, such as poly-α-olefins, for example hydrogenated or dehydrogenated polybutylenes, i.e. hydrogenated or dehydrogenated polyisobutylene.

In one non-limiting embodiment of the present disclosure, isobutylene oligomers are used. These isobutylene oligomers have a molecular weight less than 1000. In another non-limiting aspect of the present disclosure, these isobutylene oligomers are mixed with polyisobutylenes having a molecular weight that is greater than 1000. In another non-limiting embodiment, the polyisobutylenes that are mixed with the isobutylene oligomers have a molecular weight greater then 1000 and less than 10,000. In yet another non-limiting embodiment, the polyisobutylenes that are mixed with the isobutylene oligomers have a molecular weight greater then 1000 and less than 15,000.

Non-limiting examples of suitable Poly-α-olefin's include polyisobutenes commercially marketed under the trade name PERMETHYL 99 A, 101 A, 102 A, 104 A (n=16) and 106 A (n=38) by PRESPERSE Inc, or products commercially marketed under the trade name ARLAMOL HD (n=3) by ICI (where n is the polymerization level), and hydrogenated or dehydrogenated polydecenes, such as those commercially marketed for under the trade names ETHYLFLO by ETHYL CORP., and ARLAMOL PAO by ICI.

Non-limiting examples of esters that are suitable include: cetyl lactate; C₁₂-C₁₅ alkyl lactate; isostearyl lactate; lauryl lactate; linoleyl lactate; oleyl lactate; ethyl and isopropyl palmitates; C₁-C₅ alkyl myristates such as isopropyl and butyl myristate; isobutyl stearate; dioctyl malate; sebacates, such as diethyl sebacate and diisopropyl sebacate; adipates, such as diisopropyl adipate, di-n-propyl adipate, dioctyl adipate, diisostearyl adipate, maleates, such as dioctyl maleate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; dicaprylate, propylene glycol dicaprate; triisopropyl citrate; triisostearyl citrate; glyceryl trilactate; trioctyldodecyl citrate; and trioleyl citrate.

Non-limiting examples of suitable waxes include: Carnauba wax; Candelila wax; ozokerite; olive wax; rice wax; hydrogenated jojoba wax; floral absolute waxes such as blackcurrant flower essential wax commercially marketed by BERTIN (France); cerabellina; and marine waxes such as the one commercially marketed by SOPHIM under the reference M82.

Non-limiting examples of pearlescent pigments that are suitable include: titanium coated mica or bismuth oxichloride; colored pearlescent pigments such as titanium mica with avec iron oxides; titanium mica, for example, with iron blue or chromium oxide; and titanium mica with an organic pigment.

Non-limiting examples of suitable anti-dandruff agents include: selenium disulfides; hydroxypyridones and salts thereof, including Octopirex; zinc pyrithione; and climbazole.

Minoxidil and aminexil are non-limiting examples of active agents for combating hair loss.

Ethylene di-amine tetra-acetic acid (EDTA), diethylenetriamine pentacetic acid and salts thereof are non-limiting examples of sequestering agents.

Sorbic acid and salts thereof, phenoxyethanol, and parahydroxybenzoic acid salts are non-limiting examples of preserving agents that may be used.

Non-limiting examples of dyes that are suitable include: nitrated benzenic neutral; acidic or cationic direct dyes; azoic neutral; acidic or cationic direct dyes; quinone direct dyes and in particular anthraquinone neutral; acidic or cationic direct dyes; azine direct dyes; triarylmethane direct dyes; indoamine direct dyes; and natural direct dyes.

Non-limiting examples of suitable benzene type direct dyes include the following compounds:
1,4-diamino-2-nitrobenzene
1-amino-2 nitro-4-β-hydroxyethylaminobenzene
1-amino-2 nitro-4-bis(β-hydroxyethyl)-aminobenzene
1,4-bis(β-hydroxyethylamino)-2-nitrobenzene
1-β-hydroxyethylamino-2-nitro-4-bis-(β-hydroxyethylamino)-benzene
1-β-hydroxyethylamino-2-nitro-4-aminobenzene
1-β-hydroxyethylamino-2-nitro-4-(ethyl)(β-hydroxyethyl)-aminobenzene
1-amino-3-methyl-4-β-hydroxyethylamino-6-nitrobenzene
1-amino-2-nitro-4-β-hydroxyethylamino-5-chlorobenzene
1,2-diamino-4-nitrobenzene
1-amino-2-β-hydroxyethylamino-5-nitrobenzene
1,2-bis-(β-hydroxyethylamino)-4-nitrobenzene
1-amino-2-tris-(hydroxymethyl)-methylamino-5-nitrobenzene
1-hydroxy-2-amino-5-nitrobenzene
1-hydroxy-2-amino-4-nitrobenzene
1-hydroxy-3-nitro-4-aminobenzene
1-hydroxy-2-amino-4,6-dinitrobenzene
1-β-hydroxyethyloxy-2-β-hydroxyethylamino-5-nitrobenzene
1-methoxy-2-β-hydroxyethylamino-5-nitrobenzene
1-β-hydroxyethyloxy-3-methylamino-4-nitrobenzene
1-β,γ-dihydroxypropyloxy-3-methylamino-4-nitrobenzene
1-β-hydroxyethylamino-4-β,γ-dihydroxypropyloxy-2-nitrobenzene
1-β,γ-dihydroxypropylamino-4-trifluoromethyl-2-nitrobenzene
1-β-hydroxyethylamino-4-trifluoromethyl-2-nitrobenzene
1-β-hydroxyethylamino-3-methyl-2-nitrobenzene
1-β-aminoethylamino-5-methoxy-2-nitrobenzene
1-hydroxy-2-chloro-6-ethylamino-4-nitrobenzene
1-hydroxy-2-chloro-6-amino-4-nitrobenzene
1-hydroxy-6-bis-(β-hydroxyethyl)-amino-3-nitrobenzene
1-β-hydroxyethylamino-2-nitrobenzene
1-hydroxy-4-β-hydroxyethylamino-3-nitrobenzene.

Non-limiting examples of azo type direct dyes that are suitable include the cationic azo dyes described in patent applications WO 95/15144, WO 95/01772 and EP-714954. Among these compounds, non-limiting mention may be made of:
1,3-dimethyl-2-[[4-(dimethylamino)phenyl]azo]-1H-Imidazolium chloride,
1,3-dimethyl-2-[(4-aminophenyl)azo]-1H-imidazolium chloride,
1-methyl-4-[(methylphenylhydrazono)methyl]-pyridinium methyl sulfate.

Additional non-limiting examples of azo type direct dyes include those azo type direct dyes described in the Colour Index International, 3rd edition, including:
Disperse Red 17
Acid Yellow 9
Acid Black 1
Basic Red 22
Basic Red 76
Basic Yellow 57
Basic Brown 16
Acid Yellow 36
Acid Orange 7
Acid Red 33
Acid Red 35
Basic Brown 17
Acid Yellow 23
Acid Orange 24
Disperse Black 9.

Also suitable are -1-(4′-aminodiphenylazo)-2-methyl-4-bis-(β-hydroxyethyl) aminobenzene and -4-hydroxy-3-(2-methoxyphenylazo)-1-naphtalene sulfonic acid

Non-limiting examples of suitable quinone type direct dyes include:
Disperse Red 15
Solvent Violet 13
Acid Violet 43
Disperse Violet 1
Disperse Violet 4
Disperse Blue 1
Disperse Violet 8
Disperse Blue 3
Disperse Red 11
Acid Blue 62
Disperse Blue 7
Basic Blue 22
Disperse Violet 15
Basic Blue 99
1-N-methylmorpholiniumpropylamino-4-hydroxyanthraquinone
1-aminopropylamino-4-methylaminoanthraquinone
1-aminopropylaminoanthraquinone
5-β-hydroxyethyl-1,4-diaminoanthraquinone
2-aminoethylaminoanthraquinone
1,4-bis-(β,γ-dihydroxypropylamino)-anthraquinone.

Non-limiting examples of suitable azine dyes include:
Basic Blue 17
Basic Red 2.

Non-limiting examples of triarylmethane dyes that are suitable include:
Basic Green 1
Acid blue 9
Basic Violet 3
Basic Violet 14
Basic Blue 7
Acid Violet 49
Basic Blue 26
Acid Blue 7

Non-limiting examples of indoamine dyes that are suitable include:
2-β-hydroxyethlylamino-5-[bis-(β-4′-hydroxyethyl)amino]anilino-1,4-benzoquinone
2-β-hydroxyethylamino-5-(2′-methoxy-4′-amino)anilino-1,4-benzoquinone
3-N-(2′-chloro-4′-hydroxy)phenyl-acetylamino-6-methoxy-1,4-benzoquinone imine
3-N-(3′-chloro-4′-methylamino)phenyl-ureido-6-methyl-1,4-benzoquinone imine
3-[4′-N-(ethyl-carbamyl methyl )-amino]-phenyl-ureido-6-methyl-1,4-benzoquinone imine

Non-limiting examples of natural type direct dyes that are suitable include lawsone, juglone, alizarine, purpurine, carminic acid, kermesic acid, purpurogalline, protocatechaldehyde, indigo, isatine, curcumine, spinulosine, and apigenidine. Extracts or decoctions containing these natural dyes may also be used, including plasters or henna-based extracts.

Oxidation dyes, such as bases and coupling agents are also suitable dyes for use herein.

Suitable oxidation bases include paraphenylene diamines, paraamino phenols, orthoamino phenols and heterocyclic bases.

Suitable oxidation coupling agents include metadiphenols, metaaminophenols, metaphenylene diamines, naphthols and heterocyclic coupling agents.

The at least one non-polymeric cosmetic active agent is included in an amount from 0.001 to 30% by weight relative to the total weight of the at least one reducing composition. In another non-limiting embodiment, the at least one non-polymeric cosmetic active agent is present in an amount from 0.1 to 10% by weight relative to the total weight of the at least one reducing composition.

As disclosed above, the at least one reducing composition used herein comprises at least one reducing agent selected from thiol group containing compounds, which may be used in their salt form.

Non-limiting examples of thiol group containing compounds that may be included as at least one reducing agent in the at least one reducing composition include: cysteine and derivatives thereof (i.e. N-acetylcysteine); cysteamine and derivatives thereof, including C₁-C₄ acylated derivatives thereof (i.e. N-acetyl cysteamine and N-propionyl cysteamine); thiolactic acid and esters thereof (i.e. glycerol monothiolactate); thioglycolic acid and esters thereof (i.e. glycerol or glycol monothioglycolate); or thioglycerol.

Further non-limiting examples of thiol group containing compounds for use in the reducing composition include sugar N-mercapto-alkyl amides (i.e N-(mercapto-2-ethyl)gluconamide, β-mercaptopropionic acid, and derivatives thereof), thiomalic acid, pantheteine, N-(mercaptoalkyl)-ω-hydroxyalkyl amides (i.e. those described in patent application EP-A-354 835), N-mono- or N,N-dialkylmercapto 4-butyramides (i.e. those described in patent application EP-A-368 763), aminomercaptoalkyl amides (i.e. those described in patent application EP-A-432 000), alkylaminomercaptoalkyl amides (i.e. those described in patent application EP-A-514 282), and a mixture of hydroxy-2-propyl thioglycolate (2/3) and hydroxy-2 methyl-1 ethyl thioglycolate (67/33) (i.e. as described in patent application FR-A-2 679 448).

In one non-limiting embodiment, the at least one reducing composition contains from 0.1-30% by weight of the thiol group containing compounds, relative to the total weight of the at least one reducing composition. In other non-limiting embodiments, the at least one reducing composition may contains from 0.5 to 20%, such as from 1% to 10%, or, for example, less than 5% by weight of the thiol group containing compounds, relative to the total weight of the at least one reducing composition.

The at least one reducing composition pH is generally between 2 and 13, and may be between 6 and 10. In one non-limiting aspect of the present disclosure, the pH of the reducing composition is less than or equal to 9.

The pH of the at least one reducing composition may be adjusted by means of an alkaline agent, such as ammonia, organic amines such as monoethanolamine, diethanolamine, triethanolamine, 1,3-propanediamine and 2-amino-2-methyl-1-propanol, alkaline or ammonium carbonate or bicarbonate, organic carbonate such as guanidine carbonate, alkaline hydroxide or by means of an acidifying agent such as, for example, hydrochloric acid, acetic acid, lactic acid, oxalic acid or boric acid.

The at least one reducing composition typically contains at least one cosmetically acceptable solvent, such as: water; C₁-C₆ alcohols, including alkanols such as ethanol, propanol, isopropanol; polyhydric alcohols, such as propyleneglycol, pentanediol and glycerine, benzyl alcohol; polyol ethers; C₂-C₆ esters; N-methylpyrrolidone (NMP); and C₃-C₆ cetones.

The at least one reducing composition may be formed as a thickened or non-thickened lotion, a cream, a gel, or foam.

In one non-limiting embodiment of the present disclosure, applying the at least one reducing composition disclosed above represents the first step of a method for treating hair fibers.

In another non-limiting aspect of the present disclosure, the at least one reducing composition is applied onto wet and clean hair fibers.

Once the at least one reducing composition has been applied, it may be left on the hair fibers, optionally under a drying helmet, for 5 to 60 minutes. In one aspect of the present disclosure, the reducing composition is left on the hair fibers, optionally under a drying helmet, for between 5 and 30 minutes.

In one non-limiting embodiment of the present disclosure, following the application of the at least one reducing composition to the hair fibers and the passage of time, the hair is optionally rinsed. After rinsing, the hair fiber temperature is raised with a heating iron at a temperature of at least 60° C. The iron may be applied proceeding by successive separated touches during about a few seconds or by gradually moving or sliding the iron along the hair locks.

As used herein, ‘iron’ refers to a heating device which functions by contacting the hair fibers. The end of the iron coming into contact with the hair may have various forms. According to one embodiment, the iron may have a plane surface and may be referred to as a flat iron. It may also have a rounded surface which then defines a round iron. All types of flat or round irons may be used. Non-limitating examples of suitable irons for use in the method according to the invention, including those described in U.S. Pat. Nos. 4,103,145, 4,308,878, 5,983,903, 5,957,140, 5,494,058 and 5,046,516.

In other non-limiting embodiment of the present disclosure, the hair fiber is raised with a heating iron at a temperature ranging from 60° C. to 250° C., for example, from 120° C. to 220° C.

In another non-limiting embodiment, the hair fibers are not rinsed before the temperature raising step.

According to another non-limiting embodiment of the present disclosure, the method of treating hair fibers may also comprise an additional step of partially drying the hair fibers before the temperature raising step, so as to prevent steam development that might burn the hands of the hair stylist and the scalp of the user. This pre-drying step may be accomplished via a hair drier, a hood, a Climazon, or through air drying.

Another aspect of the present disclosure is to use the previously described method to durably change the hair shape without excessively altering the hair color and/or without excessively damaging the hair fibers.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contains certain errors necessarily resulting from the standard deviation found in their respective testing measurements.

The following example is intended to illustrate the invention in a non-limiting manner.

### EXAMPLE

The hair fiber treating method according to the invention was carried out using a reducing composition containing the following:

| | |
|---|---|
| Thioglycolic acid | 1.1 g |
| Niacinamide (vitamin PP or B3) | 0.1 g |
| 2-amino-2-methyl-1-propanol | qs pH 9.5 |
| Demineralized water | qs 100 g |

The reducing composition was applied onto the hair and left on for 10 minutes.

The hair was then partially pre-dried using a hair drier before being treated using a flat iron heated to 180° C.

As a result, the hair fiber showed a good texture, a well-controlled volume, and a good respect of the color. The effect of the hair treatment was long lasting and durable.

What is claimed is:

1. A hair fiber treating method without hair fixing step, comprising:
applying at least one reducing composition containing no ceramide onto hair fibers, said at least one reducing composition comprising at least one reducing agent and at least one non-polymeric cosmetic active agent, wherein said at least one reducing agent is chosen from thiol group containing compounds;
raising the temperature of the hair fibers with a heating iron at a temperature of at least 60° C., wherein the temperature of the hair fibers is raised before or after the hair fibers are optionally rinsed.

2. The hair fiber treatment method of claim 1, wherein the reducing composition does not comprise dithiodiglycolic acid or any salt thereof.

3. The hair fiber treatment method of claim 1, wherein the at least on non-polymeric cosmetic active agent is selected from: waxes; anionic, cationic, non-ionic, amphoteric or zwitterionic surfactants; active agents combating hair loss; anti-dandruff agents; sequestering agents; opacifying agents; dyes; sunscreen agents; vitamins; provitamins; fatty acids; fatty alcohols; esters; pearlescent pigments; mineral, vegetable or synthetic oils; fragrances; and preserving agents.

4. The hair fiber treatment method of claim 1, wherein said vitamins are selected from vitamins A, B3, B5 C, E and F, and said provitamins are provitamin B5

5. The hair fiber treatment method of claim 1, wherein the at least one non-polymeric cosmetic active agent is contained in an amount from 0.001 to 30% by weight relative to the total weight of the at least one reducing composition.

6. The hair fiber treatment method of claim 1, wherein the at least one non-polymeric cosmetic active agent is contained in an amount from 0.1 to 10% by weight relative to the total weight of the at least one reducing composition.

7. The hair fiber treatment method of claim 1, wherein the at least one reducing agent is contained in an amount from 0.1 to 30% by weight relative to the total weight of the at least one reducing composition.

8. The hair fiber treatment method of claim 7, wherein the at least one reducing agent is contained in an amount from 0.5 to 20% by weight relative to the total weight of the at least one reducing composition.

9. The hair fiber treatment method of claim 8, wherein the at least one reducing agent is contained in an amount from 1.0 to 10% by weight relative to the total weight of the at least one reducing composition.

10. The hair fiber treatment method of claim 1, wherein the at least one reducing agent is contained in an amount that is less than 5% by weight relative to the total weight of the at least one reducing composition.

11. The hair fiber treatment method of claim 1, wherein the thiol group containing compounds are selected from: cysteine and derivatives thereof; cysteamine and derivatives thereof; thiolactic acid and esters thereof; and thioglycolic acid and esters thereof.

12. The hair fiber treatment method of claim 11, wherein said cysteine and cysteine derivatives are N-acetylcysteine; said cysteamine and cysteamine derivatives are C₁-C₄ acylated and derivatives thereof; said thiolactic acid and esters thereof are glycerol monothiolactate; and said thioglycolic acid and esters thereof are selected from glycerol, glycol monothioglycolate, or thioglycerol.

13. The hair fiber treatment method of claim 12, wherein said cysteamine and cysteamine derivatives are selected from N-acetyl cysteamine and N-propionyl cysteamine.

14. The hair fiber treatment method of claim 1, wherein the thiol group containing compounds are used in their salt form.

15. The hair fiber treatment method of claim 1, wherein the at least one reducing composition comprises at least one solvent selected from: water; C₁-C₆ alcohols; polyhydric alcohols; benzyl alcohol; polyol ethers; C₂-C₆ esters; N-methylpyrrolidone; and C₃-C₆ cetones.

16. The hair fiber treatment method of claim 15, wherein said C₁-C₆ alcohols are alkanols, and said polyhydric alcohols are selected from propyleneglycol, pentanediol and glycerine.

17. The hair fiber treatment method of claim 16, wherein said alkanols are selected from ethanol, propanol, and isopropanol.

18. The hair fiber treatment method of claim 1, wherein the at least one reducing composition is in the form of an optionally thickened lotion, a cream, a gel, or foam.

19. The hair fiber treatment method of claim 1, wherein the hair fiber temperature is raised with a heating iron at a temperature ranging from 60° C. to 250° C.

20. The hair fiber treatment method of claim 19, wherein the hair fiber temperature is raised with a heating iron at a temperature ranging from 120° C. to 220° C.

21. The hair fiber treatment method of claim 1, wherein the at least one reducing composition is applied onto wet and clean hair fibers.

22. The hair fiber treatment method of claim 1, where once the at least one reducing composition has been applied, it is left on for a period of 5 to 60 minutes.

23. The hair fiber treatment method of claim 1, wherein the hair fibers are not rinsed prior to raising the hair fiber temperature.

24. The hair fiber treatment method of claim 1, further comprising partially pre-drying the hair fibers prior to raising the hair fiber temperature.

25. The hair fiber treatment method of claim 1, wherein the method durably changes the shape of the hair without excessively altering the hair color and/or without excessively damaging the hair fibers.
